(19) United States
(12) Patent Application Publication
(10) Pub. No.: US 2023/0285976 A1
(43) Pub. Date: Sep. 14, 2023

(54) INTERNET OF THINGS-BASED PORTABLE MULTIPLEX DIGITAL POLYMERASE CHAIN REACTION SYSYEM

(71) Applicants: SOGANG UNIVERSITY RESEARCH & BUSINESS DEVELOPMENT FOUNDATION, Seoul (KR); bioTNS Co., Ltd, Daejeon (KR)

(72) Inventors: Bong Geun CHUNG, Seoul (KR); Ji Wook CHOI, Seoul (KR); Bong Suk KIM, Daegu (KR)

(21) Appl. No.: 17/973,666

(22) Filed: Oct. 26, 2022

(30) Foreign Application Priority Data
Mar. 14, 2022 (KR) 10-2022-0031615

(51) Int. Cl. B01L 7/00; H04W 4/80; B01L 3/00

(52) U.S. Cl. CPC B01L 7/52; H04W 4/80; B01L 3/502707; G16Y 40/20

(57) ABSTRACT

Disclosed are an IoT-based portable dPCR system and a plasmonic heating module included therein, wherein the IoT-based portable dPCR system is a field-deployable diagnostic technique against the worldwide spread of infectious diseases, such as coronavirus, and is capable of detecting a plurality of viruses at once and being field-deployed through smartphone-based operation.

### CROSS-REFERENCE TO RELATED PATENT APPLICATION

This application claims priority from Korean Patent Application No. 10-2022-0031615, filed on 14 Mar. 2022 in the Korean Intellectual Property Office, the disclosure of which is incorporated herein by reference in its entirety.

### FIELD

The present disclosure relates to an Internet of Things (I)-based portable digital polymerase chain reaction (dPCR) system and, specifically, to an IoT-based portable dPCR system, which is a field-deployable diagnostic technique against the worldwide spread of infectious diseases such as coronavirus, capable of detecting a plurality of viruses at once and being field-deployed through smartphone-based operation, and to a plasmonic heating module included in the system.

### BACKGROUND

Digital polymerase chain reaction (dPCR), which is a kind of molecular diagnosis, is a third-generation polymerase chain reaction (PCR) and used for diagnosis of various diseases, such as cancer, leukemia, and various respiratory diseases. Unlike existing first-generation PCR and second-generation real-time PCR, dPCR can perform absolute quantitative analysis without a standard material or control material and is receiving attention as a next-generation diagnostic system due to its high accuracy and sensitivity.

In the event of a pandemic disease such as coronavirus, it is urgent to track and quarantine persons that may have a chance of contact with an infected person to prevent the spread of the disease. Internet of Things (IoT) technology, one of the core technologies in the era of the 4th industrial revolution, can play roles, such as remote diagnosis and data collection and processing, by combining with a bio-diagnostic system.

To determine the relative amounts of nucleic acids (genes) to be detected, digital PCR performs quantification by measuring the intensity of fluorescence after a specific amplification cycle and then comparing resultant values with quantified standard materials. Such digital PCR has advantages of absolute quantification of samples per se through the amplification of nucleic acids to be detected and good reproducibility and high sensitivity.

However, as for digital PCR, conventional techniques, such as DNA solution division through oil emulsion, are recognized to have restrictions, such as high cost incurred in the process of packaging of products and a long detection time required in the division technique. Therefore, the development to overcome the restrictions of the digital PCR are continuously conducting.

Conventionally released dPCR systems aim to detect single target DNA, and thus have a limitation in detecting mutations, such as the delta mutation or lambda mutation of coronavirus. Moreover, such systems are difficult to utilize on site due to the complicated use, high cost, and high power consumption.

Systems need to be urgently developed that can overcome the technical drawbacks of the conventional dPCR systems to diagnose multiple mutants at once and attain convenient digital PCR analysis by incorporation of the IoT technology.

### SUMMARY

The present inventors endeavored to implement a system capable of diagnosing multiple mutants at once and attaining convenient digital PCR (dPCR) analysis by the integration of IoT technology.

As a result, the present inventors confirmed that an IoT-based portable dPCR system including a self-fractionation microfluidic chip, a plasmonic heating module, a multiplex fluorescence imaging module, a digital analysis module, and a smartphone application can diagnose multiple mutants at once and attain convenient digital PCR analysis by integration of IoT technology.

An aspect of the present disclosure is to provide an IoT-based portable dPCR system.

Another aspect of the present disclosure is to provide a heating module for dPCR.

The present disclosure is directed to an IoT-based portable dPCR system including a self-fractionation microfluidic chip, a heater located configured to perform heating to allow a nucleic acid disposed in the microfluidic chip to be amplified, an image capturer configured to capture fluorescence images of the microfluidic chip to detect the amplified nucleic acid; and an analyzer configured to analyze the fluorescence image, whereby the present disclosure can easily prepare dPCR samples through the self-fractionation microfluidic chip, quickly operate at low power a temperature cycle accompanied by PCR through the heater, detect nine target DNAs through the image capturer at once, and then quantitatively diagnose the target DNAs through the analyzer.

Hereinafter, the present disclosure will be described in more detail.

An exemplary embodiment of the present disclosure is directed to an IoT-based portable dPCR system, including: a microfluidic chip configured to amplify a nucleic acid; a heater located at one side of the microfluidic chip and configured to perform heating to allow a nucleic acid disposed in the microfluidic chip to be amplified; an image capturer located at the other side of the microfluidic chip and configured to capture fluorescence images of the microfluidic chip to detect the amplified nucleic acid.

In the present disclosure, the microfluidic chip may be used for nucleic acid amplification.

In the present disclosure, the microfluidic chip may include: a fluid injection part having an inlet into which a fluid is injected; a channel part including microstructures and microchannels and communicating with the fluid injection part via a fluid entry channel through which a fluid is movable; and a discharge part communicating with the channel part and having an outlet through a fluid is discharged.

In the present disclosure, the fluid may be a hydrophilic fluid containing a nucleic acid sample or the like and a hydrophobic fluid containing an oil or the like, but is not limited thereto.

In the present disclosure, the hydrophobic fluid may be at least one selected from the group consisting of fluorocarbon, hydrofluorocarbon, a mineral oil, a silicon oil, and a hydrocarbon oil and, may be for example a mineral oil, but is not limited thereto.

In the present disclosure, the boiling point of the hydrophobic fluid may be 120 to 150° C., 120 to 145° C., 120 to 140° C., 120 to 135° C., 120 to 130° C., or 120 to 125° C., and for example, 120 to 125° C., but is not limited thereto, and a fluid having a boiling point in a range at which it does not evaporate may be used in consideration of reaction conditions of the polymerase chain reaction.

In the present disclosure, the viscosity of the hydrophobic fluid may be 0.01 to 0.15 poise, 0.01 to 0.14 poise, 0.01 to 0.13 poise, 0.01 to 0.12 poise, 0.01 to 0.10 poise, 0.01 to 0.09 poise, 0.01 to 0.08 poise, 0.01 to 0.07 poise, 0.01 to 0.06 poise, or 0.01 to 0.05 poise, and for example, 0.01 to 0.05 poise, but is not limited thereto. A viscosity of more than 0.15 poise may cause unstable sample fractionation.

In the present disclosure, the channel part may include: a first channel element communicating with the fluid entry channel; a second channel element communicating with the discharge part; and a microchannel element communicating between the first channel element and the second channel element, but is not limited thereto.

In the present disclosure, the microchannel element may include a microstructures and microchannels, but is not limited thereto.

In the present disclosure, the microchannel element may include partitioning channels each including a microstructure at one side thereof and proceeding channels, but is not limited thereto.

In the present disclosure, the partitioning channels and the proceeding channels are alternately arranged to communicate with each other, but is not limited thereto.

In the present disclosure, the microstructure may include a chamber or a chamber entry, but is not limited thereto.

In the present disclosure, a fluid may move from a partitioning channel to a chamber or a proceeding channel via a chamber entry, but is not limited thereto.

In the present disclosure, the chamber entry may include a curved portion, but is not limited thereto.

In the present disclosure, the shape of the curved portion may be a semicircle with a concave curved line, but is not limited thereto.

In the present disclosure, the width of the chamber entry may be smaller than the diameter of the chamber.

In the present disclosure, the radius of curvature of the semicircle including a concave curved line may be 25 to 150 μm, 25 to 125 μm, 25 to 100 μm, 25 to 75 μm, or 25 to 50 μm, and for example, 25 to 50 μm, but is not limited thereto.

In the present disclosure, the chamber may have an inner space, and thus for the polymerase chain reaction, a hydrophilic fluid containing a nucleic acid sample or the like can enter the inner space, but is not limited thereto.

In the present disclosure, a material of the microfluidic chip may be at least one selected from the group consisting of polydimethylsiloxane (PDMS) and an isoprene-based polymer elastomer composed of a hydrocarbon compound, and for example, polydimethylsiloxane, but is not limited thereto.

In the present disclosure, the microfluidic chip includes a microchannel including microstructures at one side thereof, so that merely an additional injection of a hydrophobic fluid, such as a mineral oil, after the injection of a sample into the microfluidic chip, can attain the fractionation of the sample into the chambers of the microstructures, and thus can prevent the evaporation and cross-contamination of the sample, but is not limited thereto.

In the present disclosure, the heater may be located at one side of the microfluidic chip to perform heating to allow a nucleic acid disposed in the microfluidic chip to be amplified, but is not limited thereto.

In the present disclosure, the heater may include: a first PCB substrate including a gold thin film layer structure on one surface thereof; a light source configured to allow a heating area of the gold thin film layer structure to undergo plasmonic heating; and a second PCB substrate configured to control the light source and a cooling fan, but is not limited thereto.

In the present disclosure, the first PCB substrate may include a gold thin film layer structure on one surface thereof, but is not limited thereto.

In the present disclosure, the first PCB substrate may further include an analog digital converter (ADC), but is not limited thereto.

In the present disclosure, the heater may further include a light source configured to allow the gold thin film layer structure of the first PCB substrate to undergo plasmonic heating, but is limited thereto.

In the present disclosure, the heater may further include a cooling fan configured to cool the gold thin film layer structure of the first PCB substrate, but is limited thereto.

In the present disclosure, the cooling fan is for cooling the gold thin film layer structure, and an example thereof is an air-cooling type cooling fan using convection or a water-cooling type cooling fan using water as a coolant, but is not limited thereto.

In the present disclosure, the cooling fan may transfer the heat of the gold thin film layer by additional connection with a material having high thermal conductivity, such as a heat pipe, but is not limited thereto.

In the present disclosure, the second PCB substrate may control the light source and the cooling fan, but is not limited thereto.

In the present disclosure, the second PCB substrate may further include pads configured to connect the light source and the cooling fan, and for example, may further include thermal pads, but is not limited thereto.

In the present disclosure, the second PCB substrate may further include a metal-oxide-semiconductor field-effect transistor (MOSFET) configured to control the light source and the cooling fan, but is not limited thereto.

In the present disclosure, the light source may allow the gold thin film layer structure of the first PCB substrate to undergo plasmonic heating by emitting light in a direction not facing the first PCB substrate. For example, the light source separately has a light reflector, so that the light emitted from the light source may be reflected through the light reflector to reach the gold thin film layer structure, but is not limited thereto.

In the present disclosure, the light source may be used for allowing a heating area of the gold thin film layer structure to undergo plasmonic heating.

In the present disclosure, the gold thin film layer structure may include fine metal wires having a winding pattern, but is not limited thereto.

In one embodiment of the present disclosure, in cases where the light source further includes the light reflector, the angle of the light source forming with the first PCB substrate, with respect to the angle of the light emitted from the light source facing the first PCB substrate through the light reflector, may be greater than 0 degrees and less than 180 degrees, for example, 90 degrees, but is not limited thereto.

In one embodiment of the present disclosure, the heater may include a light reflector, and thus the arrangement of the light source and the first PCB substrate may be changed. Such an arrangement can minimize the heater including the light reflector and focus the light source, thereby ultimately promoting the minimization of the whole IoT-based portable dPCR system according to the present disclosure, the integration of the light source and/or the convenience in system designing.

In the present disclosure, the image capturer may be located at the other side of the microfluidic chip to capture fluorescence images of the microfluidic chip in order to detect the amplified nucleic acid.

In the present disclosure, the image capturer may include a camera module, a light emitting diode (LED) light source for image capturing, an excitation filter, and an emission filter, but is not limited thereto.

In the present disclosure, the camera module may be a complementary metal-oxide semiconductor (CMOS) camera module, but is not limited thereto.

In the present disclosure, a light emitting diode (LED) light source for image capturing may be at least one selected from the group consisting of a light emitting diode having a red color (Red LED), a light emitting diode having a green color (Green LED), a light emitting diode having a blue color (Blue LED), and a light emitting diode having an amber color (Amber LED), and for example, may include all of the light emitting diodes having four red, green, blue, and yellow colors, but is not limited thereto.

In the present disclosure, the light emitted from the light emitting diode (LED) light source for image capturing, with respect to a reflection light reflected from the microfluidic chip, so as to enter the camera module, may form an angle of 40 to 60 degrees, 40 to 55 degrees, 40 to 50 degrees, 45 to 60 degrees, 45 to 55 degrees, 45 to 50 degrees, or 45 degrees, and for example, 45 degrees, but is not limited thereto.

In the present disclosure, the excitation filter may be combined with a light emitting diode (LED) light source for image capturing, but is not limited thereto.

In the present disclosure, the excitation filter may be for controlling the emission light, emitted to the microfluidic chip from the light emitting diode light source for image capturing, to have a specific wavelength, but is not limited thereto.

In the present disclosure, the emission filter may be combined with a camera module, but is not limited thereto.

In the present disclosure, the emission filter can control the camera module to capture an image of a reflection light having a specific wavelength in the reflection light reflected from the microfluidic chip, but is not limited thereto.

In the present disclosure, the excitation filter may include first fluorescence channels having λpeak values of 460 to 500 nm, 530 to 570 nm, and 610 to 650 nm, but is not limited thereto.

In the present disclosure, the emission filter may include second fluorescence channels having λpeak values of 500 to 540 nm, 565 to 605 nm, and 670 to 710 nm, but is not limited thereto.

In one embodiment of the present disclosure, the excitation filter may include four first fluorescence channels having λpeak values of 390 nm, 480 nm, 550 nm, and 630 nm.

In one embodiment of the present disclosure, the emission filter may include four second fluorescence channels having λpeak values of 430 nm, 520 nm, 585 nm, and 690 nm.

In the present disclosure, the analyzer may analyze fluorescence images, but is not limited thereto.

In the present disclosure, the analyzer may be based on Raspberry Pi, but is not limited thereto.

In the present disclosure, the analyzer may merge the fluorescence images captured by the image capturer into one RGB image, followed by analysis, but is not limited thereto.

In the present disclosure, the analyzer may detect one or more target DNA molecules by using variables of a normalized distance and a Hue value.

In the present disclosure, the normalized distance may mean, when the microfluidic chip includes at least one channel part, a coordinate value on the Y-axis perpendicular to the X-axis corresponding to the moving direction of a fluid passing through the first channel element, a microchannel element, and a second element in the channel part.

In the present disclosure, the normal distance may be for differentiating information detected from one or more different target DNA molecules among nucleic acid samples amplified in one or more channel parts.

In the present disclosure, the hue value may be a value that is used to determine the colors of the RGB image obtained by merging the fluorescence images, but is not limited thereto.

In the present disclosure, the analyzer may generate a first control information for controlling the driving of the heater, and for example, may control the heating temperature, heating time, and heating frequency of the heater, but is not limited thereto.

In the present disclosure, the analyzer may generate a second control information for driving control of the image capturer, but is not limited thereto.

The IoT-based portable dPCR system of the present disclosure may further include a mobile application.

In the present disclosure, the mobile application may include Bluetooth wireless communication and WiFi networking functions, but is not limited thereto.

In one embodiment of the present disclosure, the mobile application may be linked with the analyzer of the present disclosure via Bluetooth, but is not limited thereto.

In one embodiment of the present disclosure, the mobile application may be linked with a cloud server via WiFi networking, but is not limited thereto.

Another exemplary embodiment of the present disclosure is directed to a dPCR heating module, including: a first PCB substrate including a gold thin film layer structure on one surface thereof; a light source configured to allow a heating area of the gold thin film layer structure to undergo plasmonic heating; and a second PCB substrate configured to control the light source,

In the present disclosure, the light source may further include a light reflector, but is not limited thereto.

In the present disclosure, the light reflector may change the path of the light emitted from the light source by including a reflecting member, but is not limited thereto.

In the present disclosure, the light source may allow the gold thin film layer structure of the first PCB substrate to undergo plasmonic heating by emitting a light in a direction not facing the first PCB.

In the present disclosure, the second PCB substrate may further include a cooling fan mount to be connected to the cooling fan, but is not limited thereto.

According to the present disclosure, the IoT-based portable dPCR system of the present disclosure can achieve a rapid diagnosis of cancer diseases or respiratory infectious diseases such as coronavirus, as well as a convenient detection of sub-species, such as delta mutants, at once in the field.

Furthermore, the IoT-based portable dPCR system can shorten the time of digital PCR diagnosis through a plasmonic-based temperature controller with high heating efficiency and is highly portable by combination with a minimized imaging system. Furthermore, the IoT-based portable dPCR system enable even non-experts to continently perform dPCR analysis through linking with a smartphone application and thus has excellent utilization as an on-site diagnosis system.

In other words, the IoT-based portable dPCR system of the present disclosure can be widely used in various fields associated with on-site diagnosis.

### BRIEF DESCRIPTION OF THE DRAWINGS

FIG. 1 shows an IoT-based portable dPCR system of the present disclosure.

FIG. 2 is a schematic diagram showing a configuration of the IoT-based portable dPCR system of the present disclosure.

FIG. 3 shows a detailed configuration of a self-fractionation microfluidic chip that may be included in an IoT-based portable dPCR system according to an embodiment of the present disclosure.

FIG. 4 shows a diagram for fabricating a microfluidic chip according to an embodiment of the present disclosure.

FIG. 5 shows structures of microstructures and a microchannel included in a microfluidic chip according to an embodiment of the present disclosure.

FIG. 6 shows a detailed configuration of a heater included in the IoT-based portable dPCR system of the present disclosure to perform DNA amplification.

FIG. 7 shows a surface structure of a gold thin film layer structure included in the plasmonic heating module of the present disclosure.

FIG. 8 shows a detailed configuration of an image capturer included in the IoT-based portable dPCR system of the present disclosure to perform multiplex dPCR diagnosis.

FIG. 9 shows a method for analyzing fluorescence images by an analyzer included in the IoT-based portable dPCR system of the present disclosure.

FIG. 10 shows multiplex dPCR fluorescence images captured by using the IoT-based portable dPCR system of the present disclosure.

FIG. 11 is a graph showing the results of influenza virus analysis using the IoT-based portable dPCR system of the present disclosure.

FIG. 12 is a graph showing the results of Dengue virus analysis using the IoT-based portable dPCR system of the present disclosure.

FIG. 13 is a graph showing the results of Human coronavirus analysis using the IoT-based portable dPCR system of the present disclosure.

FIG. 14 shows the results of specificity verification of a multiplex dPCR system by using the IoT-based portable dPCR system of the present disclosure.

FIG. 15 shows a target virus prediction model using the IoT-based portable dPCR system of the present disclosure.

### DETAILED DESCRIPTION

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these examples are not construed to limit the scope of the present disclosure.

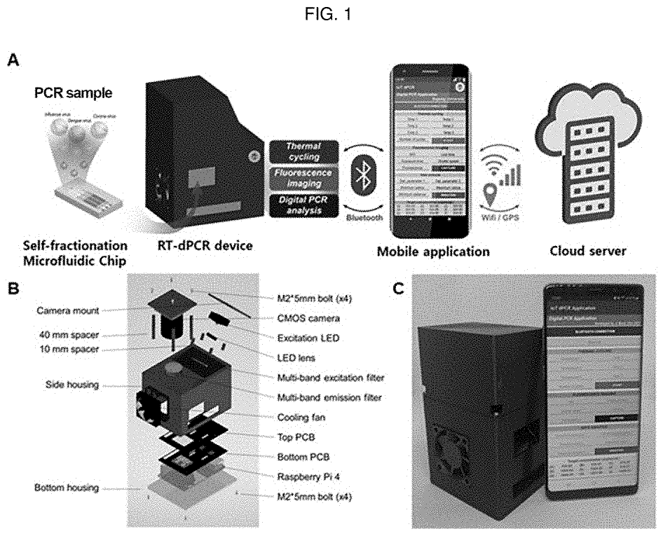

FIG. 1 shows an IoT-based portable dPCR system of the present disclosure.

Referring to FIG. 1, an IoT-based portable dPCR system may be composed of a self-fractionation microfluidic chip, a portable multiplex dPCR device, and a smartphone application. As can be seen from FIG. 1A, the IoT-based portable dPCR system of the present disclosure may include a device including a plasmonic heating module, a multiplex fluorescence imaging module, and a digital analysis module. The external appearance of the device of the present disclosure was fabricated by 3D printing and may be operated remotely via the Bluetooth connection with a developed smartphone application, but is not limited thereto. The smartphone application may be linked with a cloud server via the WIFI connection, but is not limited thereto.

The smartphone application may be developed through the Android Studio, which is a type of integrated development environment (IDE). The smartphone application may be operated as follows.
(1) The smartphone is linked with the plasmonic heating module, the multiplex fluorescence imaging module, and the digital analysis module through Bluetooth pairing.
(2) Variables necessary for DNA amplification, fluorescent image capturing, and digital analysis are transmitted to the plasmonic heating module, multiplex fluorescence imaging module, and digital analysis module.
(3) DNA amplification, fluorescent image capturing, and digital analysis are performed using the above variables.
(4) The results of multiplex dPCR analysis are converted into database files, which are then transmitted to the smartphone.
(5) The smartphone application is linked with a cloud server by input of a server address therein.
(6) The database files are transmitted to the server.
(7) The database files stored in the cloud server are downloaded to the smartphone.

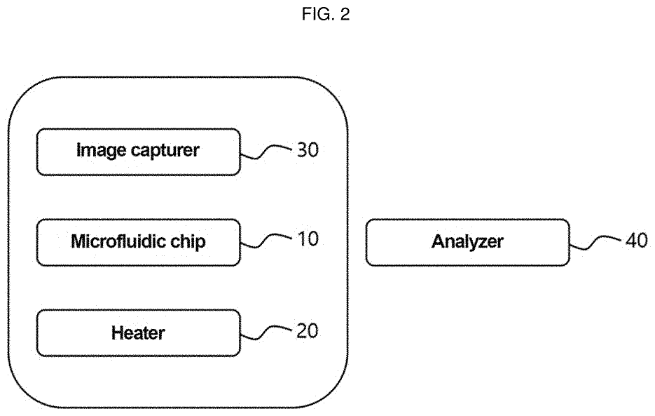

FIG. 2 is a schematic diagram showing a configuration of the IoT-based portable dPCR system of the present disclosure.

Referring to FIG. 2, the IoT-based portable dPCR system of the present disclosure may include: a microfluidic chip 10 configured to amplify a nucleic acid; a heater 20 located at one side of the microfluidic chip 10 and configured to perform heating to allow a nucleic acid disposed in the microfluidic chip 10 to be amplified; an image capturer 30 located at the other side of the microfluidic chip 10 and configured to capture fluorescence images of the microfluidic chip 10 to detect the amplified nucleic acid; and an analyzer 40 configured to analyze the fluorescence images.

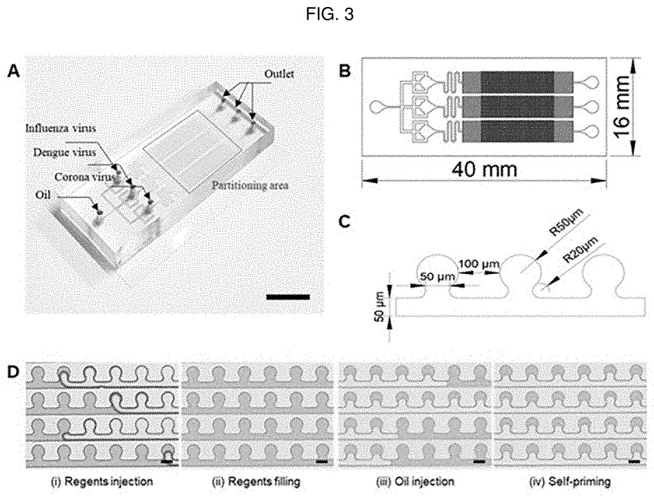

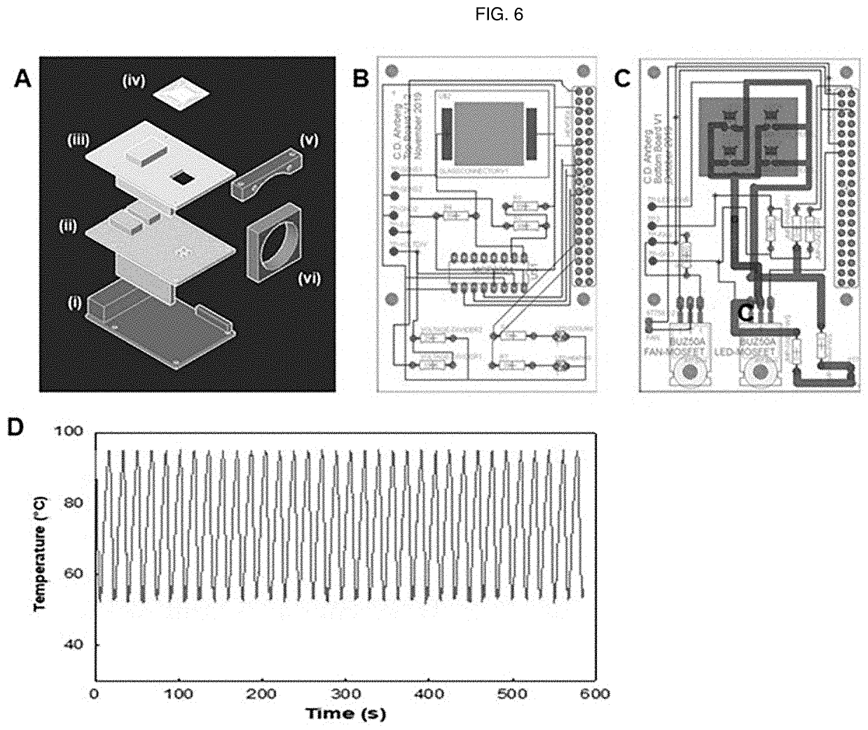

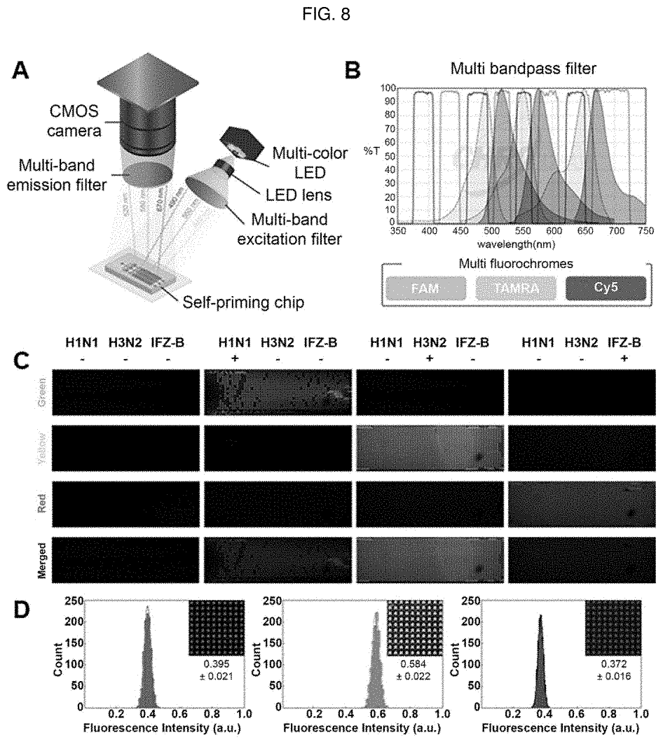

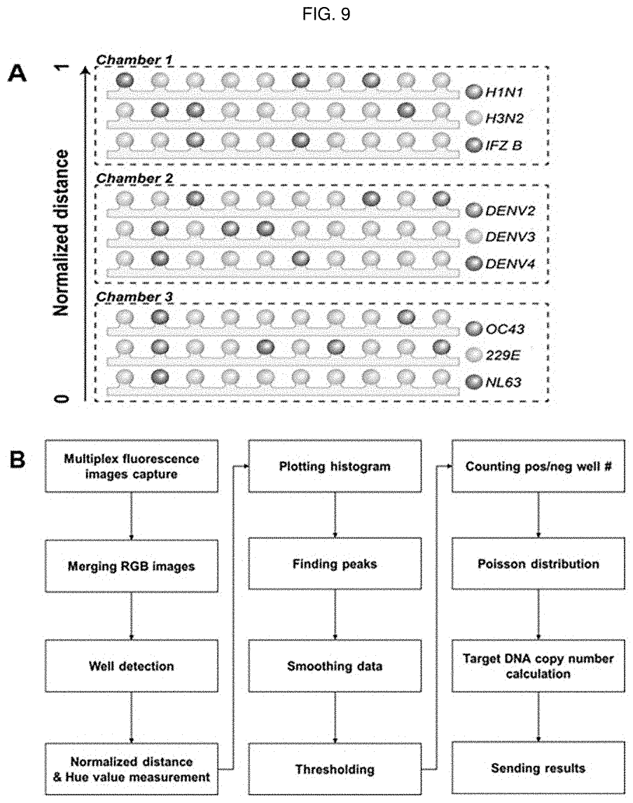

The detailed configuration of the microfluidic chip 10 is shown in FIGS. 3 to 5; the detailed configuration of the heater 20 is shown in FIG. 6; the detailed configuration of the image capturer 30 is shown in FIG. 8; and the detailed operation mechanism of the analyzer 40 is shown in FIG. 9.

FIG. 3 shows a detailed configuration of a self-fractionation microfluidic chip that may be included in an IoT-based portable dPCR system according to an embodiment of the present disclosure.

The self-fractionation microfluidic chip 10 of FIG. 3 was fabricated as follows. A silicon wafer was heated to 150° C. by a hot plate to remove water molecules remaining on the surface layer, and hexamethyldisilazane (HMDS) was applied thereon by spin coating. The surface of the wafer was washed with isopropyl alcohol (IPA) to remove unreacted HMDS and dried. A photoresist (SU8-50) was spin-coated at 1000 rpm for 30 seconds on the dried silicon wafer to form a photoresist layer with a height of 100 μm. Thereafter, the wafer was baked by heating at 65° C. and 95° C. through a hot plate.

The mask film manufactured based on the above design and the photoresist-coated wafer were placed on an aligner and exposed to ultraviolet light to transfer a pattern. The wafer was further baked through a hot plate, and unreacted photoresist was removed through a developer. The wafer was washed with isopropyl alcohol (IPA) and dried over nitrogen gas.

The fabricated wafer was hydrophobically treated with chlorotrimethylsilane, and polydimethylsiloxane (PDMS) was poured thereon, followed by baking in an oven at 80° C. for 2 hours. Inlets and outlets were formed in the PDMS mold by using a punch with a diameter of 1.5 mm, and the resultant PDMS mold, together with a glass slide, was hydrophilically treated by oxygen plasma treatment, followed by bonding, thereby fabricating the microfluidic chip of the present disclosure.

Referring to FIGS. 3B and 3C, the microfluidic chip was divided into three sections, each of which may be configured of 1,080 microwells (diameter: 100 μm) and 18 linear channels (line width: 50 μm).

As can be seen from FIG. 3D, a self-fractionation experiment of the microfluidic chip was conducted using a sample dyed with a blue dye and mineral oil. First, a sample containing a hydrophilic fluid was injected into a second fluid injection part 120 by using a pipette, and mineral oil was injected into a first fluid injection element 110, and then the results were observed through a microscope. The sample was disposed throughout the channel part 200, and thereafter, the injection of the oil resulted in successful fractionation of the sample.

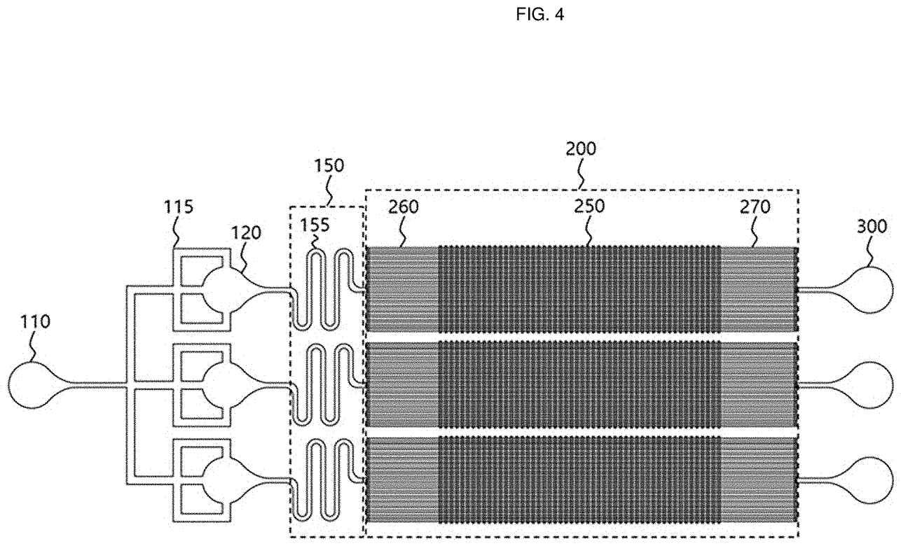

FIG. 4 shows the fabrication of such a microfluidic chip.

Referring to FIG. 4, the microfluidic chip 10 included in a system according to an embodiment of the present disclosure may include: a fluid injection part having an inlet into which a fluid is injected; a channel part including microstructures and microchannels and communicating with the fluid injection part via a fluid entry channel through which a fluid is movable; and a discharge part communicating with the channel part and having outlets through a fluid is discharged.

The fluid injection part may include an inlet through which a fluid is injected. The fluid may be a hydrophilic fluid containing a nucleic acid sample and the like and a hydrophobic fluid containing an oil and the like, but is not limited thereto.

The fluid injection part may include: a first fluid injection element 110 including a first inlet (not shown) through which a hydrophobic fluid is injected; and second fluid injection elements 120 including second inlets (not shown) through which a hydrophilic fluid is injected.

The second fluid injection elements 120 may communicate with the first fluid injection element 110 via hydrophobic fluid channels 115. The second fluid injection elements 120 may be disposed between the first fluid injection element 110 and the channel part 200.

The microfluidic chip may include at least one, two, three, or four second fluid injection elements 120, and for example, three second fluid injection elements, but is not limited thereto.

A hydrophobic fluid may be injected into the first fluid injection element 110 after a hydrophilic fluid is first injected into the second injection elements 120 to complete the injection into the microfluidic chip.

While hydrophilic fluids are respectively injected through the three second fluid injection elements 120 disposed between the first fluid injection element 110 and the channel part 200, the hydrophilic fluids injected through the three second fluid injection elements 120 may be injected without being mixed with each other when the first fluid injection element 110 is closed.

One side of the hydrophobic fluid channel 115 may communicate with the first fluid injection element 110 and the other side thereof may communicate with the second fluid injection element 120.

One side of the hydrophobic fluid channel 115 may communicate with the first fluid injection element 110 and the other side thereof may be branched into one or more branches to communicate with the second fluid injection element 120.

The other side of the hydrophobic fluid channel 115 may be branched into at least one, two, or three branches. When the other side of the hydrophobic fluid channel 115 is branched into three branches, the hydrophilic fluid remaining in the second fluid injection element 120 is easily removed by a hydrophobic fluid after the hydrophilic fluid is injected through the second injection element 120.

The hydrophobic fluid may be at least one selected from the group consisting of fluorocarbon, hydrofluorocarbon, a mineral oil, a silicon oil, and a hydrocarbon oil, and for example, may be mineral oil, but is not limited thereto.

The channel part 200 may communicate with the fluid injection part via a fluid entry channel 150 through which a fluid is movable. The channel part 200 may communicate with the second fluid injection elements 120 via the fluid entry channel 150.

The fluid entry channel 150 may include a bent portion 155, but is not limited thereto.

The number of bent portions 155 may be at least one, at least two, at least three, or at least four, and for example, may be four, but is not limited thereto.

The fluid entry channel 150 may include two or more bent portions 155, and the fluid entry channel 150 may be formed in an S shape, a zigzag shape, or a winding shape. Due to such curved portions, the fluid entry channel 150 prevents the fluid entering the channel part 200 from flowing backward and entering the second fluid injection element 120, wherein the more the bent portions 155, the more effectively the backflow can be prevented.

The channel part 200 may communicate with the fluid entry channel 150. The channel part 200 may include: a first channel element 260 communicating with the fluid entry channel 150; a second channel element 270 communicating with the discharge part 300; and a microchannel element 250 communicating between the first channel element and the second channel element.

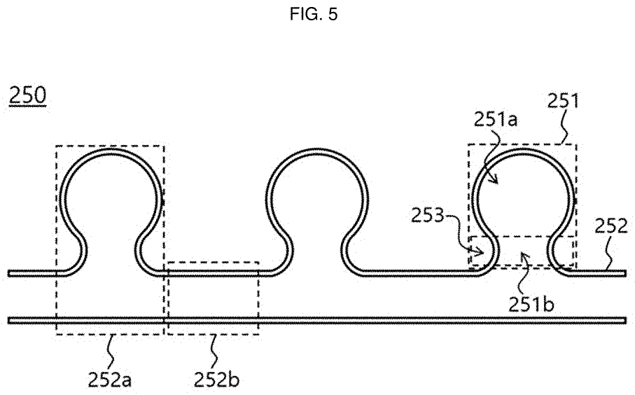

FIG. 5 shows structures of microstructures and a microchannel included in a microfluidic chip according to an embodiment of the present disclosure.

Referring to FIG. 5, the microchannel element 250 may include at least one, at least two, at least four, at least six, at least eight, at least ten, at least twelve, at least sixteen, or at least eighteen microchannels 252, and for example, may be at least eighteen microchannels 252, but is not limited thereto.

The microchannel element 250 may include microstructures 251 and a microchannel 252. The microchannel element 250 may include partitioning channels 252a each including a microstructure 251 at one side thereof and proceeding channels 252b.

The partitioning channels 252a and the proceeding channels 252b may be alternately arranged and communicate with each other. That is, the partitioning channel 252a including the microstructure at one side thereof and the proceeding channel 252b may form a unit structure, and these unit structures may be repeatedly arranged and communicate with each other. A hydrophobic fluid such as a mineral oil may be fractionated inside the microstructures by using the repeated arranged unit structures.

A fluid may move from the partitioning channel 252a to a chamber 251a or to the proceeding channel 252b through a chamber entry 251b.

The microstructure 251 may include a chamber 251a and a chamber entry 251b. The microstructure 251 may be formed in an ohm (Ω) shape.

The chamber 251a may have an inner space, and thus for the polymerase chain reaction, a hydrophilic fluid containing a nucleic acid sample and the like can enter the inner space. The chamber 251a may not allow the entry of a hydrophobic fluid into the inner space thereof. The chamber 251a may communicate with the partitioning channel 252a through the chamber entry 251b. A fluid may enter the inner space of the chamber 251a through the chamber entry 251b.

The chamber entry 251b may include a curved portion 253. The shape of the curved portion 253 may be a semicircle having a concave curved line. The width of the chamber entry 251b needs to be smaller than the diameter of the chamber 251a to effectively fractionate the sample, and the narrower the width of the chamber entry 251b, the more effectively the sample is fractionated.

The radius of curvature of the semicircle including a concave curved line may be 25 to 150 μm, 25 to 125 μm, 25 to 100 μm, 25 to 75 μm, or 25 to 50 μm, and for example, may be 25 to 50 μm, but is not limited thereto. The width rate of the chamber entry 251b and the microchannel 252 may be 1:1 to 3:1, 1:1 to 2.5:1, 1:1 to 2:1, or 1:1 to 1.5:1, and for example, may be 1:1 to 1.5:1, but is not limited thereto.

In a case where the width of the chamber entry 251b is wider than that of the microchannel 252 and thus the width ratio exceeds 3:1, the injection of a sample is difficult. On the contrary, in a case where the width of the chamber entry 251b is narrower than that of the microchannel 252 and thus the width ratio is less than 1:1, the fractionation of a sample is difficult. The width of the chamber entry 251b may mean the shortest distance among the distances between both sidewalls of the chamber entry 251b.

Since the chamber entry 251b includes a semicircle-shaped curved portion 253 including a concave curved line, the width of the chamber entry 251b may be smaller than the diameter of the chamber 251a. Therefore, when a hydrophilic fluid moves to the inner space of the chamber 251a, a hydrophobic fluid may not move to the inner space of the chamber 251a.

The diameter of the chamber 251a may be 50 to 300 μm, 50 to 250 μm, 50 to 200 μm, 50 to 150 μm, or 50 to 100 μm, and for example, may be 50 to 100 μm, but is not limited thereto.

The width of the microchannel 252 may be 20 to 100 μm, 20 to 90 μm, 20 to 80 μm, 20 to 70 μm, 20 to 60 μm, 20 to 50 μm, 30 to 100 μm, 30 to 90 μm, 30 to 80 μm, 30 to 70 μm, 30 to 60 μm, or 30 to 50 μm, and for example, may be 30 to 50 μm, but is not limited thereto.

The chamber entry 251b may prevent a hydrophobic fluid from entering the inner space of the chamber 251a.

The microfluidic chip may include a first chamber having a first diameter of a predetermined size and a second chamber having a second diameter of a predetermined size. The distance value between the centers of the first and second chambers may be greater than the sum of the first and second diameters.

The depth of the microchannel 252 and the width of the microchannel 252 may have a ratio of 1:1 to 2:1, for example, may be 2:1, but is not limited thereto. The microchannel may have an appropriate depth considering the ease of fabrication of the microfluidic chip of the present disclosure.

The fluid entry channel 150 may communicate with the microchannel element 250 via the first channel element 260. The first channel element 260 may arrange the flows of the fluids to enter the microchannel element 250. The microchannel element 250 may communicate with the discharge part 300 via the second channel element 270. The discharge part 300 may include an outlet (not shown) through which a fluid is discharged to the outside of the microfluidic chip.

FIG. 6 shows a detailed configuration of a heater included in the IoT-based portable dPCR system of the present disclosure to perform DNA amplification.

Referring to FIG. 6A, the heater may include a plasmonic heating module for plasmonic heating. The plasmonic heating module may be composed of a gold thin film layer structure that undergoes plasmonic heating, a first printed circuit board (PCB) substrate for temperature measurement and control, a second PCB substrate for operating a light emitting diode (LED) for heating and a cooling fan, a cooling fan, and a mount, and the plasmonic heating module may be derive based on Raspberry Pi.

In the drawing, (i) represents Raspberry Pi used for system control and dPCR analysis; (ii) represents a second PCB substrate for LED and cooling fan control; (iii) represents a first PCB substrate for temperature measurement of a gold thin film layer; (iv) represents a gold thin film layer structure used for plasmonic heating, (v) represents a cooling fan mount used for cooling fan fixing, and (vi) represents a cooling fan used to gold thin film layer and LED cooling.

FIG. 6B shows a detailed configuration of the first PCB substrate, and FIG. 6C shows a detailed configuration of the second PCB substrate.

The first PCB substrate may include: pads for connection of a gold thin film layer structure; Wheatstone bridges for measuring the resistance of the thin film; and an analog digital converter (ADC) calculating the temperature based on the measured resistance, but is not limited thereto.

The second PCB substrate may include: pads for connection of, for example, four light emitting diodes (LEDs) for heating and a cooling fan; and metal-oxide-semiconductor field-effect transistors (MOSFETs) for controlling the light emitting diodes (LEDs) and the cooling fan, but is not limited thereto.

As can be seen from FIG. 6D, as a result of implementing PCR thermal cycling by using the heater included in the system of the present disclosure, 35 cycles could be completed within 10 minutes.

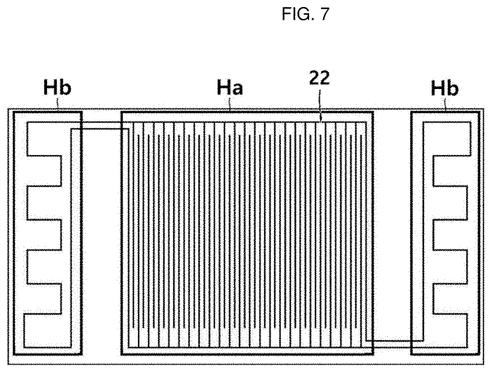

FIG. 7 shows a surface structure of a gold thin film layer structure included in the plasmonic heating module of the present disclosure.

Referring to FIG. 7A, a gold thin film layer structure according to an embodiment of the present disclosure may have fine metal wires 22 having a winding pattern that are coated with nano-sized gold (Au). The gold thin film layer structure may be divided into a heating area (Ha) that undergo plasmonic heating by the light emitting diodes for heating of the second PCB substrate and a non-heating area (Hb) that does not undergo plasmonic heating, and the fine metal wires 22 may be provided throughout the heating area (Ha) of the gold thin film layer structure.

The fine metal wires 22 according to an embodiment of the present disclosure are provided in the heating area (Ha) of the gold thin film layer structure, thereby serving as a temperature sensor for measuring the temperature of a sample. The analyzer 40 may calculate the temperature of the sample by measuring resistance information of the fine metal wires 22.

The light emitting diode light source for heating is configured to heat the fine metal wires 22, and the light source may be selected as a light source that emits wavelengths in the infrared, visible, or ultraviolet ranges, and for example, may include an LED-PCB, a diode laser array, and the like, but is not limited thereto. Any number of light emitting diodes for heating may be provided in order to emit light with an appropriate intensity. The light emitting diodes for heating may be provided together with a heat sink to thereby prepare for overheating, but is not limited thereto.

FIG. 8 shows a detailed configuration of an image capturer included in the IoT-based portable dPCR system of the present disclosure to perform multiplex dPCR diagnosis.

Referring to FIGS. 8A and 8B, the image capturer may include a multiplex fluorescence imaging module for multiplex dPCR diagnosis. The multiplex fluorescence imaging module may be composed of a CMOS camera module, an emission filter having four channels with λpeak values of 430 nm, 520 nm, 585 nm, and 690 nm, an excitation filter having four channels with λpeak values of 390 nm, 480 nm, 550 nm, and 630 nm, and a multi-color light emitting diode (LED, red, green, blue, and amber) functioning as a light source.

Specifically, the multi-color light emitting diode (LED) light source is configured to form an angle of 40 to 60 degrees with the CMOS camera module with respect to the microfluidic chip, so that the chip used for dPCR analysis could be exposed to a sufficient amount of light. Each part of the multiplex fluorescence imaging module was coupled with a mold fabricated with 3D printing, through bolts and nuts. As can be seen in FIG. 8C, images of three colors could be captured using the multi-fluorescence imaging module, and the captured images may be merged into one RGB image and then analyzed.

As can be seen in FIG. 8D, as a result of RGB image analysis, all the images of three colors captured through the multi-fluorescence imaging module showed a uniform distribution.

FIG. 9 shows a method for analyzing fluorescent images by an analyzer included in the IoT-based portable dPCR system of the present disclosure.

Referring to FIG. 9A, the analyzer may be composed of a digital analysis module for multiplex dPCR analysis, and the digital analysis module is operated through Raspberry Pi and may be based on Python. The digital analysis module can detect nine types of target DNAs through two variables (normalized distance and hue value).

In the microfluidic chip including three channel parts, the normalized distance may be used to differentiate three viruses physically separated in the three channel parts by setting the Y-axis crossing the three channel parts and deriving the coordinates on the Y-axis. The hue value may be used to differentiate subspecies for the three viruses by determining the color of the RGB images obtained by merging the images captured through the multiplex fluorescence imaging module.

Referring to FIG. 9B, multiplex fluorescence image analysis for detecting at least one virus may be performed by the following steps according to an embodiment of the present disclosure.
(1) Images of three colors are captured through the multiplex fluorescence imaging module.
(2) The captured images are merged into one RGB image.
(3) The positions and fluorescent intensities of the microstructures included in the microfluidic chip are measured through the analysis algorithm.
(4) The measured values are converted into normalized distances and hue values, separately.
(5) The converted values are plotted as a histogram.
(6) The peak values are derived from the histogram.
(7) The histogram is smoothened to remove noise.
(8) A threshold point is designed among the peak values.
(9) Positive and negative wells are differentiated based on the threshold point, and the ratio therebetween is determined.
(10) The determined ratio is calculated through the Poisson distribution.
(11) The final DNA copy number of each of nine viruses is derived.

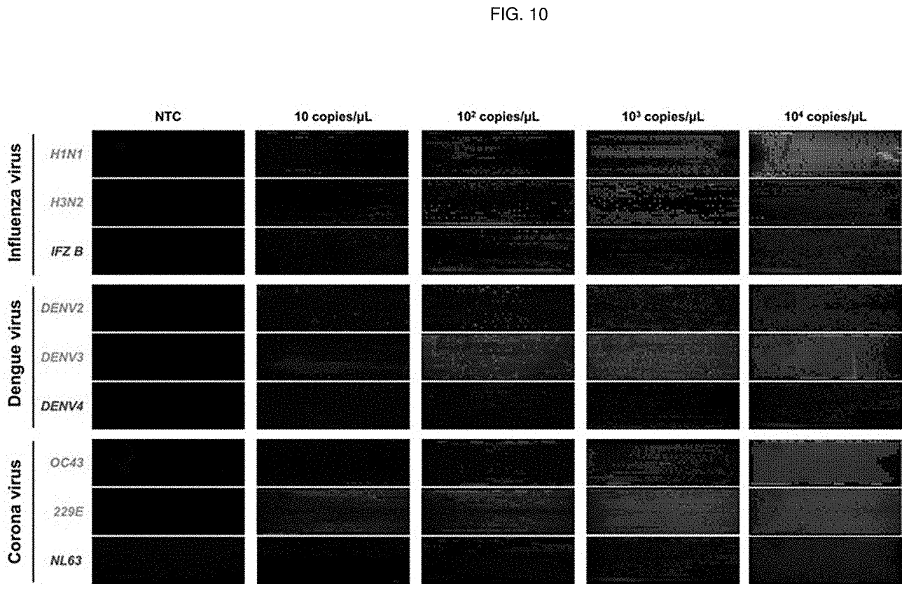

The results of experiments conducted using PCR samples of various concentrations (10 copies/μL, 100 copies/μL, 1,000 copies/μL, or 10,000 copies/μL) are shown in FIG. 10. In the experiments, the PCR samples included master mixes, forward primers, reverse primers, probes, and target DNAs. Specifically, the target DNAs were divided into three species of influenza viruses (H1N1, H3N2, and IFZ B), three species of dengue viruses (DENV2, DENV3, and DENV4), and three species of human coronaviruses (OC43, 229E, and NL63). FAM (λex=492 nm, λex=518 nm), TAMRA (λex=555 nm, λex=580 nm), and Cy5 (λex=650 nm, λex=680 nm) were used as the probes depending on the subspecies.

As can be seen in FIG. 10, as the concentration increased, the positive signal of the fluorescence images increased in each virus subspecies sample. The sensitivity of the multiplex dPCR analysis was 10 copies/μL, and the operating range was 10 to 10,000 copies/μL.

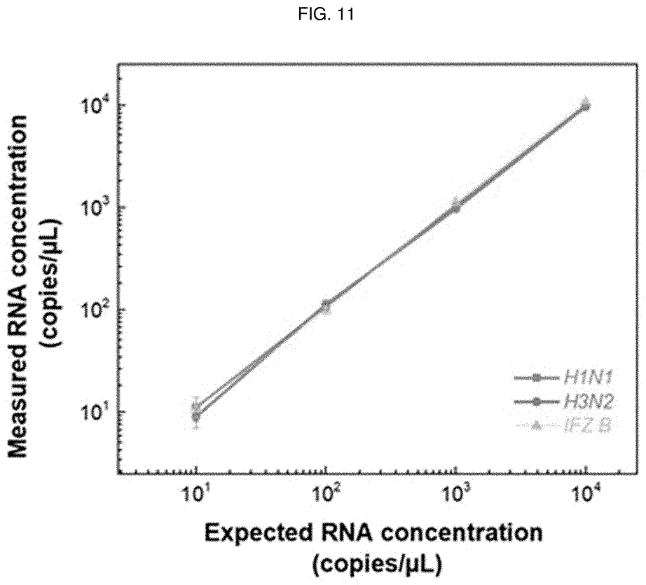

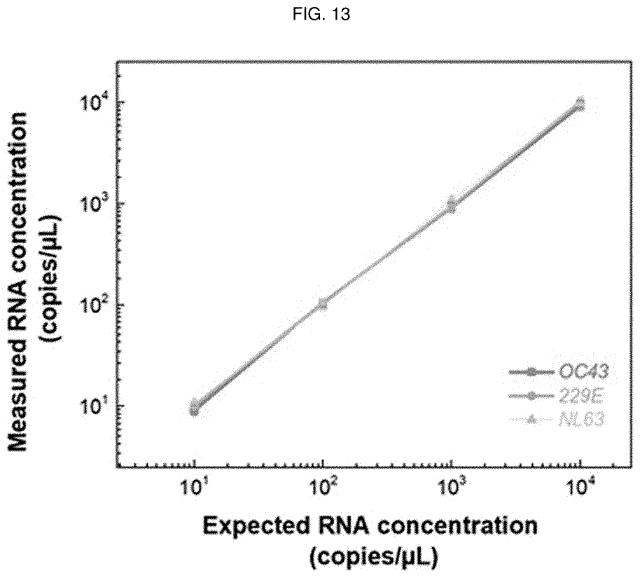

As a result of quantitative analysis based on fluorescence images, as shown in FIGS. 11 to 13, high accuracy (CV less than 10%) and high linearity (R2>0.980) were shown for the nine species of viruses.

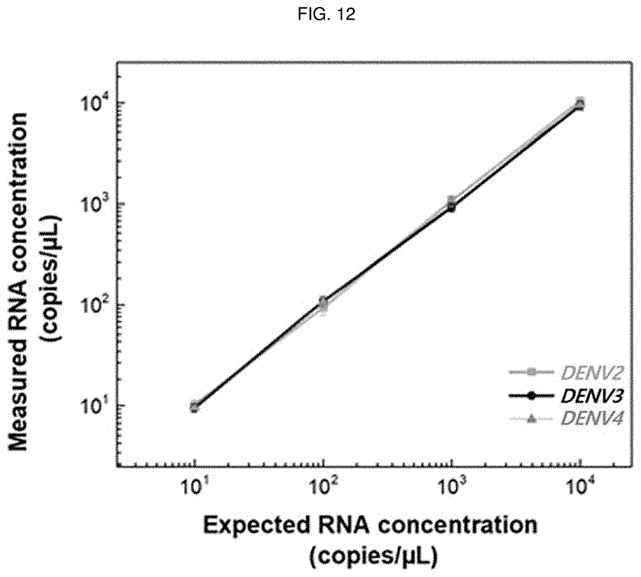

Specifically, as can be seen in FIG. 11, the linearity values of the three species of influenza viruses were H1N1 (0.984), H3N2 (0.997), and IFZ B (0.993), respectively. As can be seen in FIG. 12, the linearity values of the three species of dengue viruses were DENV2 (0.994), DENV3 (0.992), and DENV4 (0.999), respectively. As can be seen in FIG. 12, the linearity values of the three species of human coronaviruses were OC43 (0.996), 229E (0.997), and NL63 (0.990), respectively.

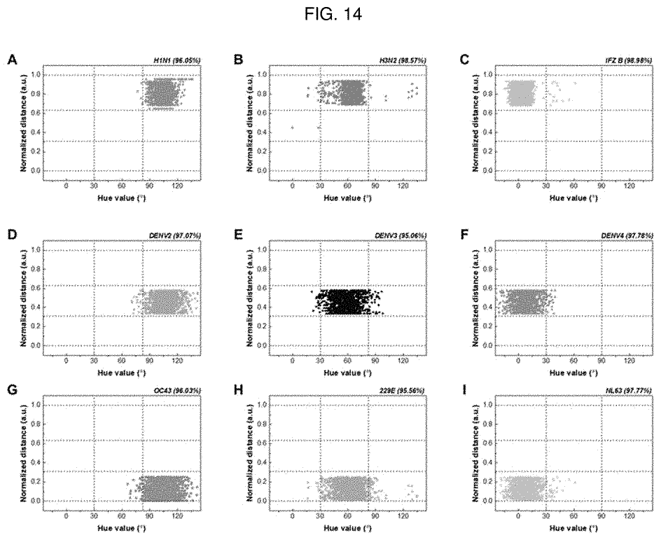

The results of specificity verification for the PCR samples are shown in FIG. 14. As for specificity verification, each of the nine species of viruses was subjected to dPCR analysis to evaluate whether corresponding viruses could be differentiated.

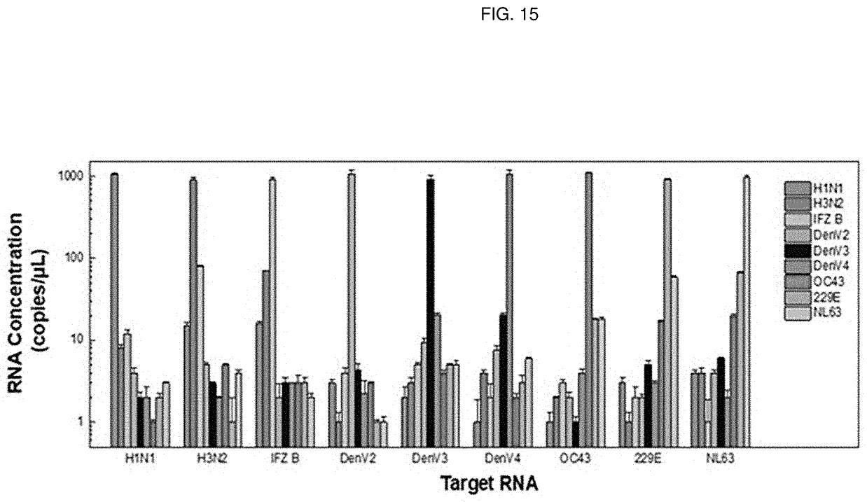

As can be seen in FIG. 14, as a result of evaluation based on the normalized distance (coordinate values on Y-axis) and hue value in the microfluidic chip with self-fractionation ability, an accuracy of 95% or more was shown for all the nine species of viruses, indicating high specificity. As a result of quantitative analysis based on the results of specificity analysis, a model for predicting target RNA of an injected virus with high accuracy was established, and these results are shown in FIG. 15.

Specifically, FIG. 15 shows that a total of nine species of target RNAs were H1N1, H3N2, IFZ B, DenV2, DenV3, DenV4, OC43, 229E and NL63 from the left, and differentially listed in the higher order of target RNA of one species in each sample.

It was consequently confirmed that the IoT-based portable dPCR system proposed in the present disclosure can be easily operated and quickly diagnose nine species of viruses with high accuracy and sensitivity.

### EXPLANATION OF REFERENCE NUMERALS

| | |
|---|---|
| 10: microfluidic chip | 20: heater |
| 22: fine metal wire | |
| Ha: heating area | Hb: non-heating area |
| 30: image capturer | 40: analyzer |
| 110: first fluid injection element | |
| 115: hydrophobic fluid channel | |
| 120: second fluid injection element | |
| 150: fluid entry channel | |
| 155: bent portion | 200: channel part |
| 250: microchannel element | 251: microstructure |
| 251a: chamber | 251b: chamber entry |
| 252: microchannel | 252a: partitioning channel |
| 252b: proceeding channel | 253: curved portion |
| 260: first channel element | 270: second channel element |
| 300: discharge part | |

What is claimed is:

1. An Internet of Things (IoT)-based portable dPCR system, comprising:
a microfluidic chip configured to amplify a nucleic acid;
a heater located at one side of the microfluidic chip and configured to perform heating to allow a nucleic acid disposed in the microfluidic chip to be amplified;
an image capturer located at the other side of the microfluidic chip and configured to capture fluorescence images of the microfluidic chip to detect the amplified nucleic acid; and
an analyzer configured to analyze the fluorescence images,
wherein the heater comprises:
a first PCB substrate comprising a gold thin film layer structure on one surface thereof;
a light source configured to allow a heating area of the gold thin film layer structure to undergo plasmonic heating; and
a second PCB substrate configured to control the light source.

2. The IoT-based portable dPCR system of claim 1, wherein the microfluidic chip comprises:
a fluid injection part having an inlet into which a fluid is injected;
a channel part comprising microstructures and microchannels and communicating with the fluid injection part via a fluid entry channel through which a fluid is movable; and
a discharge part communicating with the channel part and having an outlet through a fluid is discharged.

3. The IoT-based portable dPCR system of claim 2, wherein the fluid injection part comprises:
a first fluid injection element having a first inlet through which a hydrophobic fluid is injected; and
a second fluid injection element having a second inlet through which a hydrophilic fluid is injected,
wherein the first fluid injection element and the second injection element communicate with each other via a hydrophobic fluid channel.

4. The IoT-based portable dPCR system of claim 3, wherein one side of the hydrophobic channel communicates with the first fluid injection element and the other side thereof is branched into one or more branches to communicate with the second fluid injection element.

5. The IoT-based portable dPCR system of claim 2, wherein the microchannel element comprises partitioning channels each including a microstructure at one side thereof and proceeding channels, the partitioning channels and the proceeding channels being alternately y arranged to communicate with each other.

6. The IoT-based portable dPCR system of claim 5, wherein the microstructure comprises a chamber entry and a chamber, the chamber communicating with the partitioning channel via the chamber entry.

7. The IoT-based portable dPCR system of claim 6, wherein the chamber entry comprises a curved portion, and
wherein the width of the chamber entry is smaller than the diameter of the chamber.

8. The IoT-based portable dPCR system of claim 7, wherein the shape of the curved portion is a semicircle with a concave curved line, the radius of curvature of the semicircle being 25 to 150 μm.

9. The IoT-based portable dPCR system of claim 1, wherein a material of the microfluidic chip is at least one selected from the group consisting of polydimethylsiloxane (PDMS), polymethyl methacrylate (PMMA), and polycarbonates (PC).

10. The IoT-based portable dPCR system of claim 1, wherein the heater comprises: a first PCB substrate having a metal layer and fine metal wires located on one surface thereof; and a second PCB substrate configured to allow the metal layer and the fine metal wires to undergo plasmonic heating.

11. The IoT-based portable dPCR system of claim 1, wherein the image capturer comprises a camera module, a light emitting diode (LED) light source for image capturing, an excitation filter, and an emission filter.

12. The IoT-based portable dPCR system of claim 11, wherein a light emitted from the light emitting diode (LED) light source for image capturing forms an angle of 40 to 60 degrees with a reflection light reflected from the microfluidic chip so as to enter the camera module.

13. The IoT-based portable dPCR system of claim 11, wherein the excitation filter includes first fluorescence channels of 500 to 540 nm, 565 to 605 nm, and 670 to 710 nm.

14. The IoT-based portable dPCR system of claim 11, wherein the emission filter includes second fluorescence channels of 460 to 500 nm, 530 to 570 nm, and 610 to 650 nm.

15. The IoT-based portable dPCR system of claim 1, wherein the analyzer is based on Raspberry Pi.

16. The IoT-based portable dPCR system of claim 1, wherein the analyzer further comprises a mobile application, the mobile application including Bluetooth wireless communication and WiFi networking functions.

17. A dPCR heating module, comprising:
a first PCB substrate comprising a gold thin film layer structure on one surface thereof;
a light source configured to allow a heating area of the gold thin film layer structure to undergo plasmonic heating; and
a second PCB substrate configured to control the light source,
wherein the light source allows the gold thin film layer structure of the first PCB substrate to undergo plasmonic heating by emitting a light in a direction not facing the first PCB substrate.
